(19) United States
(12) Patent Application Publication
(10) Pub. No.: US 2024/0112028 A1
(43) Pub. Date: Apr. 4, 2024

(54) PHYSICAL AUGMENTATION OF PATIENT DATA FOR MEDICAL APPLICATIONS

(71) Applicant: Carl Zeiss Meditec AG, Jena (DE)

(72) Inventors: Hendrik Burwinkel, Oberkochen (DE); Michael Trost, Oberkochen (DE); Nicolas Bensaid, Oberkochen (DE); Stefan Saur, Oberkochen (DE)

(73) Assignee: Carl Zeiss Meditec AG, Jena (DE)

(21) Appl. No.: 18/478,048

(22) Filed: Sep. 29, 2023

(30) Foreign Application Priority Data
Sep. 30, 2022 (DE) 102022125420.8

(51) Int. Cl. G06N 3/08; G16H 10/60

(52) U.S. Cl. CPC G06N 3/08; G16H 10/60

(57) ABSTRACT

A method for training a machine learning system with an extended set of patient data is described. This method includes measuring patient data and assigning ground truth data, determining the number of data pairs E/A, determining whether the number of data pairs lies below a previously defined training data threshold value, and if this is the case, carrying out the following steps: selecting a physical-optical model; using data pairs E/A in order to determine corresponding second output vectors A″ from input vectors E by means of the relation function R, determining a respective difference vector, modifying the input vectors by an ε-vector; determining third output vectors of the relation function; determining modified output vectors; and training a machine learning system by means of the modified data and the original data.

### CROSS REFERENCE TO RELATED APPLICATIONS

This U.S. patent application is a continuation of, and claims priority under 35 U.S.C. § 119(d) to German Patent Application DE 10 2022 125 420.8, filed on Sep. 30, 2022. The disclosure of this prior application is considered part of the disclosure of this application and is hereby incorporated by reference in its entirety.

### TECHNICAL FIELD

This disclosure relates to a method for training a machine learning system, in particular to a method for training a machine learning system with an extended set of patient data. The disclosure furthermore relates to a training data generator system for training a machine learning system with an extended set of patient data, and to a computer program product.

### BACKGROUND

It is virtually unimaginable not to use camera systems in the clinical field and particularly during operations. With the use of camera systems, image processing systems have also made inroads into routine surgical practice. Image processing systems are usually based on artificial intelligence systems or machine learning systems. The functioning of such systems is very greatly dependent on the training data that are available and used. This is all the more applicable in the medical domain, since very reliable results—predictions in the technical jargon—of the machine learning systems are especially important here. At the same time, it is often very difficult to obtain a comparatively high number of similar training data attributed to measured patient data. This is also owing to the fact, inter alia, that different surgical methods are used in different hospitals, and so hospital-specific—and more often also physician-specific or operation-type-specific—training data are required. Not just in the context of comparatively rare illnesses, the provision of the necessary amount of training data may constitute a bottleneck in the successful use of machine learning systems as part of surgery assistance systems.

What happens again and again, therefore, is that low training data coverage in individual areas of a data domain results in relatively severe “overfitting” behavior of the machine learning systems. As a consequence, this normally results in unusable predictions of the machine learning systems and hence non−usability of the surgery assistance systems, which are actually highly usable and appreciated by surgeons.

For this reason, the principle of data augmentation has already been used successfully. That enables new data to be generated from known data, which can then be used for training purposes, for example by way of transformations such as rotation, translations or mirroring (tilting). This is occasionally done with image data, while numerical data may be overlaid with random noise data. Such modifications reduce the risk of an overfitting behavior because the data sets used per training epoch differ slightly.

However, such standard augmentation techniques focus only on generating data which are not recognized as already known, which would result in an overfitting behavior. Hitherto the standard augmentation techniques have not, however, integrated new information into the data, which results in new but sub−optimal data points and may reduce the performance of the respective machine learning system.

Therefore, there is a need for improved and extended data augmentation techniques in order to generate from a small number of patient data additional training data which comprise additional information by comparison with the original data.

### SUMMARY

This object is achieved by means of the method proposed here, the corresponding system and the associated computer program product in accordance with the independent claims. Further configurations are described by the respective dependent claims.

In accordance with one aspect of the present disclosure, a method for training a machine learning system with an extended set of patient data E′/A′ is presented. In this case, the method comprises measuring patient data as a plurality of data pairs E/A, wherein the data pairs can comprise input vectors E and assigned first output vectors A. The output vectors A can represent ground truth data for a machine learning system if associated input vectors E are used during a learning phase of the machine learning system.

The method furthermore comprises determining the number of data pairs of the measured patient data, and determining whether the number of data pairs lies below a previously defined training data threshold value. If that is the case, the following steps are carried out in accordance with the method:

(i) selecting a physical-optical model for a relation function R on the basis of at least one first subset of the plurality of data pairs E/A, wherein an approximation vector with respect to the associated output vector is determined by the relation function R as the result if an associated input vector E is used as the argument of the relation function R;

(ii) using at least one second subset of the plurality of data pairs E/A in order to determine corresponding second output vectors A″ from input vectors E by means of the relation function R;

(iii) determining, for all data pairs of the second subset, a respective difference vector Δ between the determined corresponding second output vector A″ of the relation function R and the associated ground truth data vector A;

(iv) modifying the input vectors E of the data pairs E/A of the second subset by a respective ε-vector, wherein the components of the ε-vector each have absolute values much smaller than the standard deviations of the components of the associated input vector relative to all the input vectors;

(v) determining third output vectors A′″ of the relation function R, wherein the modified input vectors E′ of the data pairs of the second subset are used as arguments of the relation function R; and (vi) determining modified output vectors A′ by subtracting the determined difference vectors Δ from the third output vectors A″′

Finally, the method additionally comprises training a machine learning system for generating a machine learning model by means of the modified input vectors E′, the modified output vectors A′ and the measured patient data.

In accordance with a further aspect of the present disclosure, a training data generator system for training a machine learning system with an extended set of patient data E′/A′ is also presented. In this case, the system comprises a processor and a memory which operatively cooperates with the processor to store instructions which, when executed by the processor, cause the processor to implement the following: measuring patient data as a plurality of data pairs E/A, wherein the data pairs comprise input vectors

E and assigned first output vectors A, and wherein the output vectors A represent ground truth data for a machine learning system if associated input vectors E are used during a learning phase of the machine learning system.

The processor is furthermore caused to implement determining the number of data pairs of the measured patient data, and determining whether the number of data pairs lies below a previously defined training data threshold value. If that is the case, the processor is furthermore caused to carry out the following steps:
(i) selecting a physical-optical model for a relation function R on the basis of at least one first subset of the plurality of data pairs E/A, wherein an approximation vector with respect to the associated output vector is determined by the relation function R as the result if an associated input vector E is used as the argument of the relation function R,
(ii) using at least one second subset of the plurality of data pairs E/A in order to determine corresponding second output vectors A″ from input vectors E by means of the relation function R,
(iii) determining, for all data pairs of the second subset, a respective difference vector Δ between the determined corresponding second output vector A″ of the relation function R and the associated ground truth data vector A,
(iv) modifying the input vectors E of the data pairs E/A of the second subset by a respective ε-vector, wherein the components of the ε-vector each have absolute values much smaller than the standard deviations of the components of the associated input vector relative to all the input vectors,
(v) determining third output vectors A′″ of the relation function R, wherein the modified input vectors E′ of the data pairs of the second subset are used as arguments of the relation function R, and
(vi) determining modified output vectors A′ by subtracting the determined difference vectors Δ from the third output vectors A′″.

Finally, the processor is caused to implement training a machine learning system for generating a machine learning model by means of the modified input vectors E′, the modified output vectors A′ and the measured patient data.

The proposed method for training a machine learning system with an extended set of patient data has a plurality of advantages and technical effects which may also apply accordingly to the associated system:

The inventive concept presented here firstly addresses the disadvantages of standard data augmentation techniques. In this case, new data points are generated which contain more information than originally available medical patient data.

Theory-based relations or dependencies between input parameters and target values exist for various fields of application in medical use. One example thereof is calculations for refractive indices of intraocular lenses to be inserted (IOL=intraocular lens). In this case, an optical-physical model yields a mathematical expressible relation between biometric input parameter values and a target value for the IOL refractive power.

The procedure is based on a theory-based augmentation comprising the following steps. It therefore allows measured patient data to be supplemented by additional data points containing additional information that goes beyond information contained in the measured patient data.

The steps carried out comprise here, in particular:
defining a theoretical or mathematical model which provides a relation R between input data and corresponding, associated target data for a task which is intended to be provided by the trained machine learning model;
using the available patient-based training data as arguments of the relation R, for a determination of (output) values of the relation R;
determining a difference or an offset between the determined values and current ground truth data for each of the used training data points or training data pairs which are recruited from the available patient-based training data;
performing a small modification to the available training data and a renewed determination of (output) values of the relation R for the qualified data;
subtracting the offset from the thus ascertained values of the relation R from the previous step; and
using the pair of the modified input data and the modified (output) values as additional new training data for training the machine learning model.

Since the newly generated data points are influenced by the used theory or the mathematical model of the relation R, they contain additional information that is valuable for the training of the machine learning system. What this involves, therefore, is not just a mere supplementation of the available data points by comparatively simple mathematical operations such as mirroring, rotations or translations, but rather a genuine supplementation of additional information on the basis of the optical-physical model, which clearly goes beyond a purely mathematical model.

This can be applied explicitly to cataract treatments, in which a precise refractive power of the intraocular lens to be inserted is of very great importance for the patient and their eyes. For this purpose, therefore, a physical model R describing a relation between measured patient input data and a target value, namely e.g. the refractive power of the IOL, is actually designed or selected. The patient input data here are biometric or ophthalmological measured patient data. These are used as input values for the physical model R.

The result values determined therefrom are extracted from the original output values of the training data pairs (input values/output values) and stored.

Subsequently, in an advantageous manner, the input data are then provided with small random modifications and fed again to the physical model R as input data, from which new, modified output data—that is to say target values for the refractive power of the IOL—can be determined.

Afterward, the individually determined offsets are subtracted from the newly determined target values for the refractive power of the IOL. In this way, in an advantageous manner, new data pairs thus arise for training a machine learning system for determining an IOL refractive power for an intraocular lens to be inserted, which are additionally influenced by the physical model, and thus contain new information −namely that of the optical-physical model—that goes beyond the original biometric or ophthalmological measured patient data.

In this way, the limitations of the traditional data augmentation techniques, in particular in the medical domain and even more precisely for use in cataract operations—i.e. for an even more precise prediction of a refractive power for intraocular lenses to be inserted—are overcome if machine learning systems are used here as subsystems of surgery assistance systems.

In other words, it can thus additionally be stated that by subtracting the offset from the generated result values, the modification of the result value is directly established on the basis of the associated real data pair. Consequently, the augmentation is neither a pure simulation on the basis of a physical-optical model nor purely random noise, but rather a particularly advantageous combination of both effects.

Further exemplary embodiments are presented below, which have validity both in association with the method and in association with the corresponding system.

In accordance with one advantageous embodiment of the method, components of the respective ε-vectors can be generated randomly. In this case, the data can be normalized with respect to mean value and standard deviation. In this way, by means of epsilon, it is then possible to effect a scaling in portions of the individual standard deviation. For the value epsilon=1, for example, each parameter can be varied by its individual standard deviation. Epsilon can be an ε-vector, too, in which individual elements can be zero. In this way, it is possible to influence individual incremental values for each vector dimension of the input and output vectors.

In accordance with one supplementary embodiment of the method, the machine learning system can be selected from the group consisting of a fully connected neural network system, a recurrent neural network system, a convolutional neural network system, a graph neural network system, a transformer network system and a foundation model system. Further machine learning system architectures are conceivable. In principle, the concept presented here is not restricted to specific types of learning system architectures.

In accordance with a further advantageous embodiment of the method, a subset of the data pairs can consist of biometric measurement variables of the eye and a refractive power of an intraocular lens as input vector and a refractive result of cataract surgery as output vector. In this case, these vectors would be one-dimensional vectors—i.e. essentially scalar values of one-dimensional ophthalmological data.

In accordance with another advantageous embodiment of the method, a subset of the data pairs can consist of biometric measurement variables of the eye and the refractive result of cataract surgery as input variable and the refractive power of an intraocular lens as output variable. This would constitute another type of use of the proposed concept for increasing the number of training data.

In accordance with an additional advantageous embodiment of the method, the refractive result can also represent an objective refraction; in this case, a subjective refraction can be an additional output variable. By this means, the output data would then actually be representable as a vector.

In accordance with a furthermore advantageous embodiment of the method, a subset of the data pairs can also correspond to biometric measurement variables of the eye and a refractive result of refractive surgery as input vector and an actually expected refraction as output vector. It is evident from this, too, that a plurality of possibilities for input and output values or vectors to be chosen are possible in order to realize the basic concept proposed. There would still be a prerequisite, however: A meaningful physical model would need to be available in order to map the input values to the output values. If no physical model should be designated, however, a mathematically derived model for the data pairs could also already be sufficient in order to be able to utilize the advantages of the concept presented.

In accordance with another elegant embodiment of the method, the relation function R can be selected on the basis of a subset of the first data pairs. In other words, there is not always a need for all of the measured data pairs, which results in greater flexibility of the concept proposed. It is also possible to use a plurality of subsets from the overall set of the measured patient data. In this case, the principles of ensemble learning could be used since different subsets would generate different additional training data based possibly on different physical or optical-physical (or, in the simpler case, mathematical) models.

In accordance with one extended embodiment of the method, the relation function can describe a physical dependence, a statistical correlation or a dependence corresponding to an individually trained machine learning system. Consequently, here as well there are elegant and advantageous variation possibilities within the scope of the proposed concept in order to cope with different initial situations in regard to the amount of measured patient data. In particular, depending on the type and extent of the available patient data, it is also possible to use different stored parameter values of different physical-optical models. That includes a selection from different physical-optical models. Examples in respect thereof would be a calculation using the vergence principle, a paraxial ray tracing calculation, a calculation using matrix optics or complete ray tracing.

In accordance with another further developed embodiment of the method, the relation can represent a physical-optical description of the eye. A very realistic supplementation of measured patient eye data which is oriented toward physical reality could be obtained by this means.

In accordance with one helpful embodiment of the method, the components—in particular associated absolute values—of the respective ε-vector each have absolute values much smaller—in the mathematical sense—than the standard deviation of the corresponding components of the associated input vectors or can be equal to zero. The latter would correspond to a situation in which no modification is performed for a component of the associated input vector.

The latter would apply in particular to zero components of the associated input vector since other values—in particular in terms of absolute values—cannot be smaller than a zero value. Therefore, value ranges of −1 to +1 would certainly be addressed as well.

In accordance with one extended embodiment of the method, this can additionally comprise at least one of the following: displaying the measured patient data—e.g. as measured data pairs—displaying the modified input vectors E′ and the modified output vectors A′—e.g. in a comparison—receiving a signal—if appropriate from an operator—for confirming individual vector pairs of the modified input vectors E′ and the associated modified output vectors A′ (or all simultaneously), and excluding non-confirmed vector pairs for the training of the machine learning system.

The comparison can be effected here in the form of tables or graphically, thereby making it significantly easier for an operator to recognize at a glance how the training data were extended. In this way, it is also possible to recognize, if appropriate, whether specific extensions of the potential training data in partial areas have tendencies which, in the case of a table−like representation, are ascertainable only with difficulty or not at all. In this way, it is possible to create a better user interface for the area of the extension of training data on the basis of an additional physical model (i.e. besides the measured data).

Furthermore, embodiments can relate to a computer program product able to be accessed from a computer−usable or computer−readable medium that comprises program code for use by, or in conjunction with, a computer or other instruction processing systems. In the context of this description, a computer-usable or computer-readable medium can be any device that is suitable for storing, communicating, transferring, or transporting the program code.

The details of one or more implementations of the disclosure are set forth in the accompanying drawings and the description below. Other aspects, features, and advantages will be apparent from the description and drawings, and from the claims.

### DESCRIPTION OF DRAWINGS

It should be pointed out that exemplary embodiments of the disclosure may be described with reference to different implementation categories. In particular, some exemplary embodiments are described with reference to a method, whereas other exemplary embodiments may be described in the context of corresponding devices. Regardless of this, it is possible for a person skilled in the art to identify and to combine possible combinations of the features of the method and also possible combinations of features with the corresponding system from the description above and below—if not specified otherwise—even if these belong to different claim categories.

Aspects already described above and additional aspects of the present disclosure are evident inter alia from the exemplary embodiments described and from the additional further specific embodiments described with reference to the figures.

FIG. 1 shows a flowchart−like illustration of one exemplary embodiment of the method according to the disclosure for training a machine learning system with an extended set of patient data.

FIG. 2 illustrates an eye, together with various biometric parameters of the eye.

FIG. 3 shows a flowchart−like illustration, related more closely to the implementation, of a sequence of a supplementation of measured patient data.

FIG. 4 shows a diagram of one exemplary embodiment of the training data generator system for training a machine learning system with an extended set of patient data.

FIG. 5 illustrates one exemplary embodiment of a computer system that comprises the system according to FIG. 4. Like reference symbols in the various drawings indicate like elements.

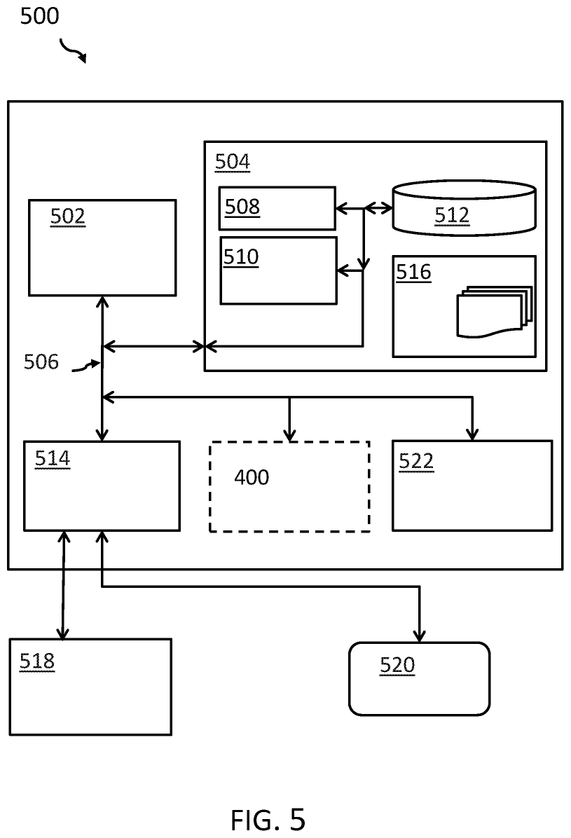

### DETAILED DESCRIPTION

In the context of this description, conventions, terms and/or expressions should be understood as follows:

The term “machine learning system” may describe a non-procedural system whose behavior in relation to input data and output data generated therefrom (or in relation to vectors in each case) is conditioned by a learning process in which example data—i.e., both input data and output data—are used. Parameter values of the learning system are optimized during the learning process and a learning model for predicting output data on the basis of unknown input data is available after the learning or training has been completed. By way of example, the optimization process can be implemented by way of a back-propagation function of differences between generated output data on desired output data. Various architectures for machine learning systems are known. Examples include a neural network and a deep neural network.

The term “extended set of patient data” may in this case describe a set of data which may be sufficient for achieving good training of a machine learning system. Patient data may be available in an insufficient amount, and so the number of these data should be increased. However, this should be done not just on an abstract mathematical function but on a relation (i.e. function) which takes account of additional physical-optical insights and/or dependencies.

The term “patient data” may in this case describe ophthalmological measurement data which are available preferably directly or in buffer-stored form—e.g., in a patient measurement data memory.

The term “ground truth data”, in the field of machine learning, describes target or output data (or vectors) to be learned if specific input data are used as a starting point. Such input and output data form associated data pairs for the training of the machine learning system.

The term “training data threshold value” may describe a predefined amount or number of data pairs which should be attained in order that training of a machine learning system can be carried out reasonably (i.e. without disturbing side effects to be expected). With fewer available data pairs (also applies to vectors as input/output data), either more data pairs have to be collected or the method proposed here is used.

The term “physical-optical model” may describe known dependencies of ophthalmological data by means of a formula or relation. Various models are known.

The term “relation function R” may describe the dependence just described between ophthalmological data values of a patient and the resulting refractive power after a cataract operation.

The term “biometric measurement variables” may relate to ophthalmological data values of a patient or else to other measured data value pairs for which a dependence function is known, which are based on known physical dependencies, but nevertheless have a variability which need not be covered 100% by the respective model.

A detailed description of the figures is given below. It is understood in this case that all of the details and instructions in the figures are illustrated schematically. A flowchart-like illustration of one exemplary embodiment of the method according to the disclosure for training a machine learning system with an extended set of patient data is illustrated first. Further exemplary embodiments, or exemplary embodiments for the corresponding system, are described subsequently.

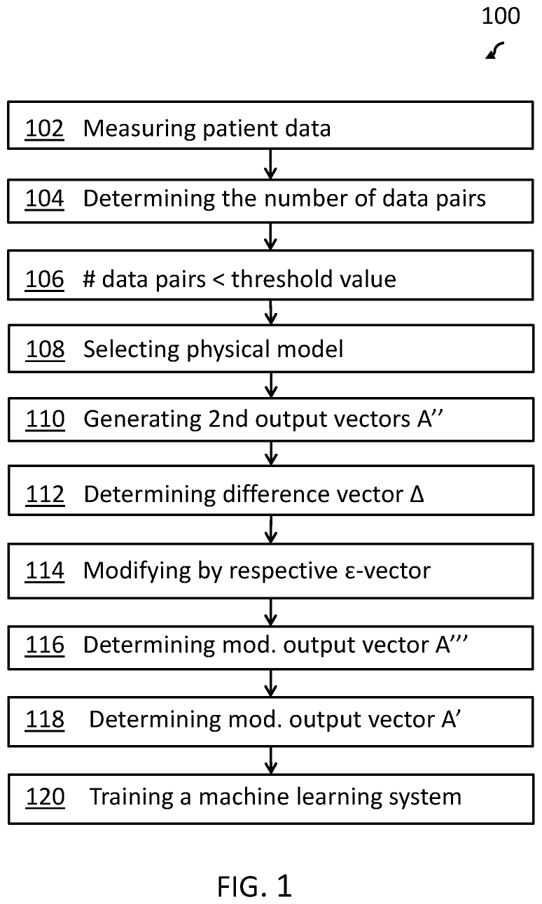

FIG. 1 shows a flowchart-like illustration of one preferred exemplary embodiment of the method 100 for training a machine learning system with an extended set of patient data E′/A′. The method comprises measuring 102 patient data—e.g. ophthalmological data—as a plurality of data pairs E-A. In this case, the input vectors E and assigned first output vectors A are assigned to one another. They can be scalar values (e.g. index of refraction) or else vectors of the measured data values. The output vectors A are then ground truth data for training of a machine learning system if associated input vectors E are used during a learning phase of the machine learning system.

The method 100 furthermore comprises determining, 104, the number of data pairs of the measured patient data, and determining, 106, whether the number of data pairs lies below a previously defined training data threshold value. Said training data threshold value can either be configured or be derived from the composition or the set of the data pairs.

The method 100 furthermore comprises selecting, 108, a physical-optical model for a relation function R on the basis of at least one first subset of the plurality of data pairs E/A. In this case, results of the relation function R can represent an approximation vector with respect to the associated output vector—i.e. quasi-ground truth vectors—if an associated input vector E is used as the argument of the relation function R. The submethod of selecting the physical-optical model is advantageously based on the ophthalmological data already available. Respective other physical-optical models—and thus other relations R—may be better suited depending on data range or value range. For this purpose, for example, a look-up table can be used. Moreover, it is possible for the selection to be based on an input parameter. The input parameter value can be input into a dialog function by a user. The same applies, mutatis mutandis, to the training data threshold value.

Furthermore, the method 100 comprises determining, 110, corresponding second output vectors A″ from input vectors E by using at least one second subset of the plurality of data pairs E/A and by means of the relation function R.

Furthermore, the method 100 comprises determining, 112, a respective difference vector Δ for all the data pairs of the second subset—either scalar values or vectors (e.g. as distance vectors)—between the determined second output vector A″ corresponding to the result of the relation function R and the associated ground truth data vector A.

Finally, the method 100 comprises modifying 114 the input vectors E of the data pairs E/A of the second subset by a respective ε-vector, wherein the components of the ε-vector have absolute values much smaller than the standard deviation of the components of the associated input vector. This can relate to the length scalar values or else respective individual components.

This is taken as a basis for determining 116 third output vectors A′″ of the relation function R, wherein the modified input vectors E′ of the data pairs of the second subset are used as arguments of the relation function R, and furthermore determining 118 modified output vectors A′ by subtracting the determined difference vectors Δ from the third output vectors A′″

Finally, the method 100 is supplemented by training 120 a machine learning system for generating a machine learning model by means of the modified input vectors E′, the modified output vectors A′ as additional data pairs and the measured patient data.

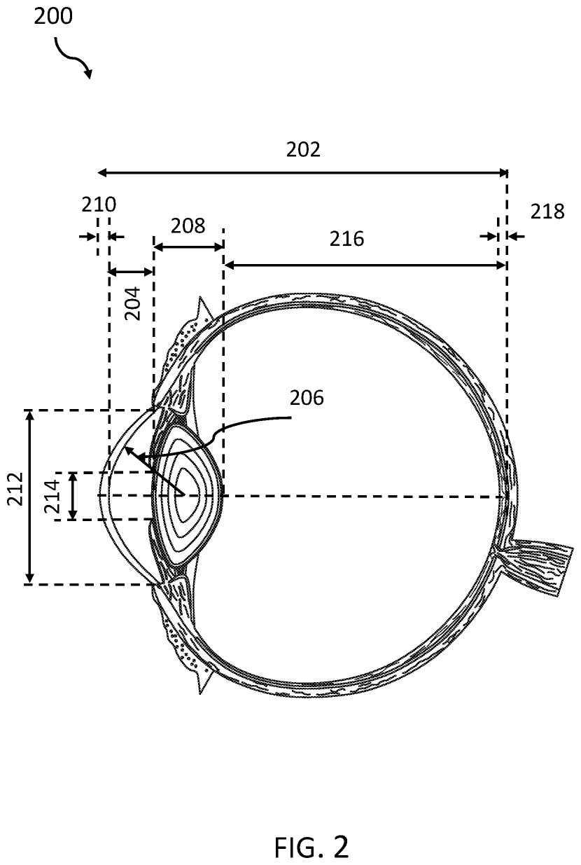

FIG. 2 illustrates an eye 200 with various biometric parameters of the eye. In particular, the following parameters are illustrated: axial length 202 (AL), anterior chamber depth 204 (ACD), keratometry value 206 (K, radius), refractive power of the lens, lens thickness 208 (LT), central cornea thickness 210 (CCT), white-to-white distance 212 (WTW), pupil size 214 (PS), posterior chamber depth 216 (PCD), retina thickness 218 (RT). At least one of these parameters is contained both in the ophthalmological training data and in the ophthalmological data of a patient, which are each contained in the subject matter of the concept presented here. All these potential measurement variables and further measurement data values can be used as input data for the method mentioned above.

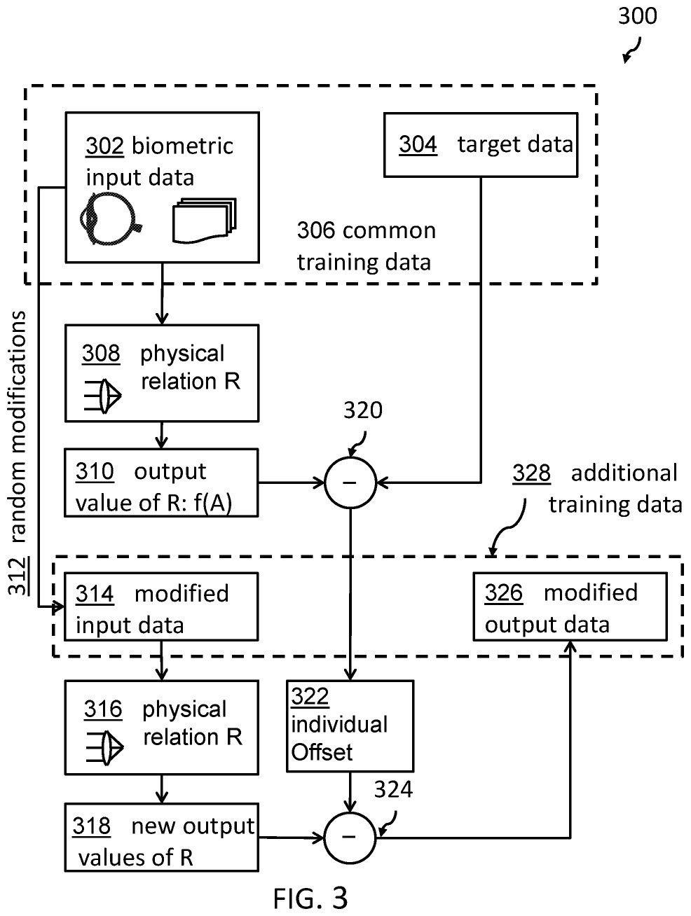

FIG. 3 shows a flowchart—like illustration of an embodiment 300 of the proposed method that is related more closely to the implementation. As an initial scenario, it may be imagined that a machine learning system is intended to be trained, but not enough training data are available. In the present case, the training data are represented as common training data 306 composed of measured biometric input data 302 and associated target data 304 as ground truth data. Vectors of the input data and output data or else scalar values can be involved in each case.

The first step—once it has been established that not enough training data are available—consists in ascertaining, 308, a physical correlation R from the available measurement biometric input data 302 and the target data 304. At this juncture, additional physical dependencies influence the entire method, such that the additionally generated training data are not generated at a purely abstract level, but rather take account of additional physical dependencies.

In the next step, 310, output values (or vectors) are determined from the entire set of the measured biometric input data 302, or a subset thereof, by means of the ascertained physical relation R.

In a partly parallel process branch, the measured biometric input data 302 are provided with random modifications 312 (i.e. ε-values which each have absolute values much smaller than the standard deviations of the corresponding measured input data; in the case of a vector, either the length of the vector or the respective component of the vector). On the basis of these modified input data 314, new output values 318 are generated by means of the physical relation R, 316.

Once again in parallel, individual offset values 322 for the target data are ascertained from the output values 310 and the original target data 304 by means of the difference unit 320. Modified output data 326 can then be generated by means of a further difference unit 324. In this way, what is produced as additional training data 328 is the set of the modified input data 314 and the output data 326 which were generated and modified in the manner just described.

This exemplary embodiment just specified will now be concretised further again. As is known, in the context of cataract operations, with the aid of intraocular lens calculation formulae for ocular biometry for a patient, a selected intraocular lens refractive power is predicted, namely what refractive result should be expected after insertion of the intraocular lens. This traditionally takes place using a physical-optical formula—i.e. e.g. by means of the relation R. One example of such a physical-optical formula would be a paraxial ray tracing approach. However, similar results can also be achieved by means of a machine learning system or a machine learning model, in which case the machine learning system then “acts like the formula”, i.e. produces comparable results. For this purpose, the machine learning system has to be trained by means of data points (multidimensional data points), the ocular biometry and the IOL refractive power serving as input data for the machine learning model and the refractive result (i.e. the resulting refractive power after the operation) representing the ground truth data or the label of the data point.

As already mentioned above, the number of real measured medical data (here ophthalmological data) may be too small to provide a sufficient data set for the training of a machine learning system for creating a machine learning model. In accordance with the method and corresponding system proposed here, with the aid of the physical-optical augmentation, from a real data point, a new additional data point is now generated which can likewise be used for the training and which comprises physical-optical additional information in comparison with traditional augmentation. Said information is added by or by way of the physical-optical function or relation.

This substantive matter will now be elucidated by means of a concrete numerical example. According to the disclosure, the following takes place in a first step: Measuring patient data as a plurality of data pairs E-A, wherein the data pairs comprise input vectors E and associated first output vectors A, wherein the output vectors A represent ground truth data for a machine learning system if associated input vectors E are used during a learning phase of the machine learning system. It should be pointed out once again here that the data pairs should be understood as data points which can each comprise a plurality of components, i.e. multidimensional data points. This will also become apparent from the following numerical example.

It shall be assumed that the following data point was measured and an extended data point is intended to be augmented therefrom. As biometric ophthalmological data, for example, the following values are acquired as input vector E:
axial length: 24.3 mm;
anterior chamber depth: 2.7 mm;
lens thickness: 4.5 mm;
cornea thickness: 0.55 mm;
keratometry: 42.5 diopters

An IOL refractive power of 20 diopters is assumed as refractive power for the intraocular lens (IOL).

A refractive result of −0.25 diopters is obtained as the result or output vector A.

In this case, the method step “determining the number of data pairs of the measured patient data” would thus establish that one data pair/data point is present, wherein the input data are predefined by the vector of the biometric-ophthalmological data indicated above and the result value is represented by the refractive result of −0.25 diopters.

In the method step “determining whether the number of data pairs lies below a previously defined training data threshold value”, it is established that in this example only one data pair is present, which will definitely not be sufficient for the training. The question of whether enough data pairs are present can be settled by checking a predefined threshold value.

A further method step follows, namely “selecting a physical-optical model for a relation function R on the basis of at least one first subset of the plurality of data pairs E/A, wherein an approximation vector with respect to the associated output vector is determined by the relation function R as the result if an associated input vector E is used as the argument of the relation function R”. In this case, an appropriately matching relation function R which matches the measured data pairs already present is selected from a plurality of possible relation functions R. Predefined selection criteria can be stipulated for this. In this case, the relation function R selected is a known IOL calculation formula for ocular biometry for a patient which predicts a resulting IOL refractive power for a selected intraocular lens (IOL). By way of example, a paraxial ray tracing formula can be selected. With the aid of this formula or relation function R, the entry of the input vector can then be converted into a physical calculation of an expected output value. This calculation constitutes the physical predication or prediction of the refractive result and thus the approximation of the actual output vector.

This is followed by a further step of the proposed method, namely “using at least one second subset of the plurality of data pairs E/A in order to determine corresponding second output vectors A” from input vectors E by means of the relation function R″. Consequently, the paraxial ray tracing formula—i.e. the selected relation function R which is now concretized—is applied to the input vector E in order to determine a physical predication or prediction for the IOL refractive power for a cataract operation, namely the output vector A″, which here is just a one-dimensional vector which can be represented as a scalar value.

To continue the above example, it is now possible to use the input vector E
axial length: 24.3 mm;
anterior chamber depth: 2.7 mm;
lens thickness: 4.5 mm;
cornea thickness: 0.55 mm;
keratometry: 42.5 diopters
IOL refractive power: 20 diopters

in order to calculate the refractive result.

There subsequently follows a further sub-step of the proposed method, namely “calculating the output vector A″ by means of the physical-optical ray tracing model”, i.e. the relation function R (that is to say the selected ray tracing formula). In this case, an expected post-operative refractive result of −0.4 diopters results, for example.

Finally, the method step “determining, for all data pairs of the second subset, a respective difference vector Δ between the determined corresponding second output vector A″ of the relation function R and the associated ground truth data vector A” is carried out. In other words: The ground truth data vector A is subtracted from the predicted output vector A″ in order to obtain the difference vector Δ. This results in the difference vector

Δ=−0.4D−(−0.25D)=−0.15D.

In the further method step “modifying the input vectors E of the data pairs E/A of the second subset by a respective ε-vector, wherein the components of the ε-vector each have absolute values much smaller than the standard deviations of the components of the associated input vector relative to all the input vectors”, for example, the following ε-vector can be generated randomly (e.g. by means of a pseudorandom generator):

### Biometry

ε axial length: 0.2 mm;
ε anterior chamber depth: −0.1 mm;
ε lens thickness: 0.3 mm;
ε cornea thickness: 0.0 mm;
ε keratometry: −0.2 diopters.

A value of e.g. −0.3 diopters can be assumed as the value for the final refractive power of the IOL. As is evident, the magnitude of each of the ε-components is at most 10% that of the original values. This estimated value can also be used for the definition of “each . . . much smaller than the standard deviations of the components of the associated input vector”. Of course, the respective ε-components can also be significantly less than 10% of the initial values.

By adding this ε-vector to the initial input vector E, the following new modified input vector E′ is then generated:

### Biometry

axial length: 24.5 mm;
anterior chamber depth: 2.6 mm;
lens thickness: 4.8 mm;
cornea thickness: 0.55 mm;
keratometry: 42.3 diopters;

### IOL

IOL refractive power: 19.7 diopters

In this way, already in the next method step “determining third output vectors A′″ of the relation function R, wherein the modified input vectors E′ of the data pairs of the second subset are used as arguments of the relation function R”, by means of inserting the modified input vector E′ into the relation function R—i.e. the ray tracing formula—it is possible to calculate e.g. the following output vector A″′ on the basis of the physical-optical dependencies as the refractive result of −0.2 diopters.

Therefore, the modified output vector A′ then also arises by way of:

−0.2D−(−0.15D)=−0.05D, i.e. −0.05 diopters.

These value pairs can then be used in implementing the final method step “training a machine learning system for generating a machine learning model by means of the modified input vectors E′, the modified output vectors A′ and the measured patient data”. Consequently, besides the original data pairs E/A, the physically augmented data pairs E′/A′ are now also used for the training of the machine learning system. For the sake of completeness, these data shall be mentioned here once again:

### Modified Input Vector E′

### Biometry

axial length: 24.5 mm;
anterior chamber depth: 2.6 mm;
lens thickness: 4.8 mm;
cornea thickness: 0.55 mm;
keratometry: 42.3 diopters;

### IOL

IOL refractive power: 19.7 diopters; modified output vector A′ (here actually only a scalar value):

Refractive result after inserting the IOL: −0.05 diopters.

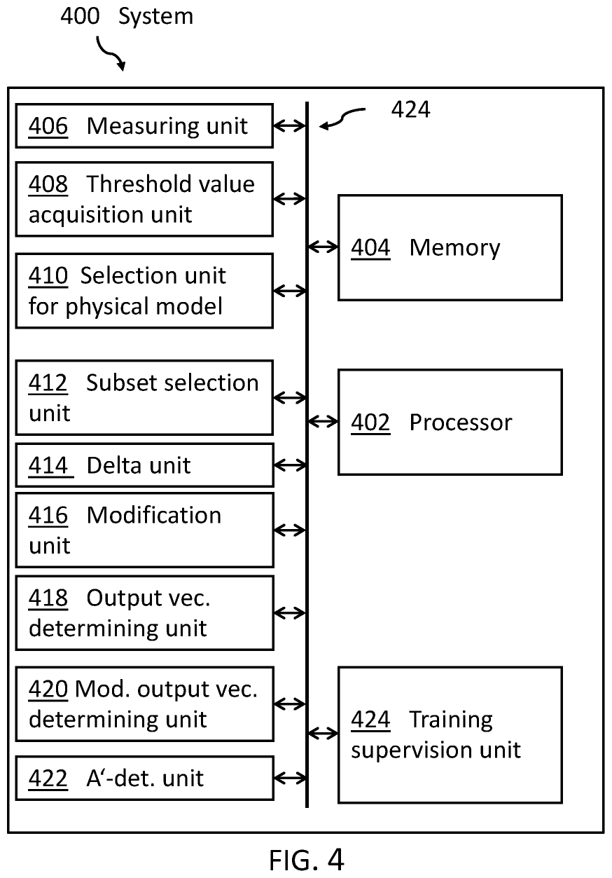

FIG. 4 shows one exemplary embodiment of the training data generator system 400 for training a machine learning system with an extended set of patient data E′/A′. The system comprises a processor 402 and a memory 404 which operatively cooperates with the processor 402 to store instructions which, when executed by the processor 402, cause the processor 402 to implement measuring patient data as a plurality of data pairs E/A, wherein the data pairs comprise input vectors E and assigned first output vectors A, wherein the output vectors A represent ground truth data for a machine learning system if associated input vectors E are used during a learning phase of the machine learning system. Measuring the patient data—in particular ophthalmological measurement data—can preferably be effected by means of an OCT scan (OCT=optical coherence tomopgraphy). In this case, in ophthalmology, an imaging method can be used to obtain two- and three-dimensional recordings of scattering materials (for example biological tissue) with micrometer resolution. In ophthalmology, OCT is used to detect spatial differences in the reflection behavior of individual retinal layers, and morphological structures can be represented with a high resolution. Generally, the measuring is performed by the measuring unit 406.

Alternatively or supplementarily, “A-scan” (axial depth scan) or “B-scan” methods can also be used in order to measure the patient data. An A-scan describes a one-dimensional result of a scan of a patient's eye, which describes information about geometric dimensions and locations of structures within the eye (cf. FIG. 2). In contrast thereto, a B-scan describes a lateral overlay of a plurality of the aforementioned A-scans, to obtain a section through the eye. By combining a plurality of layers of the eye generated in this way, volume views are also generable and measurement data are generable. As a further method for ascertaining measurement data of a patient, an “en-face OCT” method would also be conceivable, which is known as a method for producing transverse sectional images of the eye—in contrast to longitudinal sectional images by way of the abovementioned A- or B-scans.

The processor 402 is furthermore caused to implement determining the number of data pairs of the measured patient data, for example by means of a patient data counting device.

In addition, the processor 402 is caused to determine—with the aid of a threshold value acquisition unit 408—whether the number of data pairs lies below a previously defined training data threshold value. If that is the case, the processor is caused to carry out the following steps:
(i) selecting—e.g. by means of a selection unit 410 for a physical model—a physical-optical model for a relation function R on the basis of at least one first subset of the plurality of data pairs E/A, wherein an approximation vector with respect to the associated output vector is determined by the relation function R as the result if an associated input vector E is used as the argument of the relation function R;
(ii) using at least one second subset of the plurality of data pairs E/A in order to determine corresponding second output vectors A″ from input vectors E by means of the relation function R, e.g. by means of a subset selection unit 412;
(iii) determining, for all data pairs of the second subset, a respective difference vector Δ between the determined corresponding second output vector A″ of the relation function R and the associated ground truth data vector A, e.g. by means of a delta unit 414 designed for determining the difference vector Δ;
(iv) modifying the input vectors E of the data pairs E/A of the second subset by a respective ε-vector, wherein the components of the ε-vector each have absolute values much smaller than standard deviations of the associated input vectors, e.g. by means of a modification unit 460 configured precisely for this purpose;
(v) determining third output vectors A′″ of the relation function R, wherein the modified input vectors E′ of the data pairs of the second subset are used as arguments of the relation function R, e.g. by means of an output vector determining unit 418; and
(vi) determining modified output vectors A′ by subtracting the determined difference vectors Δ from the third output vectors A′″, e.g. by means of an A′ determining unit 420.

In addition, the processor 402 is furthermore caused to implement training a

machine learning system—in particular by means of a training supervision unit 422—for generating a machine learning model by means of the modified input vectors E′, the modified output vectors A′ and the measured patient data.

It should be expressly pointed out that the modules and units—in particular the processor 402, the memory 404, the measuring unit 406, the threshold value acquisition unit 408, the selection unit for the physical model 410, the subset selection unit 412, the delta unit 414, the modification unit 416, the output factor determining unit 418, the modifying output vector determining unit 420, the A′ determining unit 420, and the training supervision unit 422—can be connected to electrical signal lines or via a system-internal bus system 424 for the purpose of signal or data exchange.

The computer system 500 has a plurality of general-purpose functions. The computer system may in this case be a tablet computer, a laptop/notebook computer, some other portable or mobile electronic device, a microprocessor system, a microprocessor-based system, a smartphone, a computer system with specially configured special functions, or else a constituent part of a microscope system. The computer system 500 may be configured so as to execute computer system-executable instructions—such as for example program modules—that may be executed in order to implement functions of the concepts proposed here. For this purpose, the program modules can comprise routines, programs, objects, components, logic, data structures etc. in order to implement particular tasks or particular abstract data types.

The components of the computer system can comprise the following: one or more processors or processing units 502, a storage system 504 and a bus system 506 that connects various system components, including the storage system 504, to the processor 502. The computer system 500 typically comprises a plurality of volatile or non-volatile storage media accessible by the computer system 500. The storage system 504 may store the data and/or instructions (commands) of the storage media in volatile form—such as for example in a RAM (random access memory) 508—in order to be executed by the processor 502. These data and instructions realize one or more functions and/or steps of the concept presented here. Further components of the storage system 504 may be a permanent memory (ROM) 510 and a long-term memory 512, in which the program modules and data (reference sign 516) and also workflows may be stored.

The computer system comprises a number of dedicated devices (keyboard 518, mouse/pointing device (not illustrated), screen 520, etc.) for communication purposes. These dedicated devices can also be combined in a touch-sensitive display. An I/O controller 514, provided separately, ensures a frictionless exchange of data with external devices. A network adapter 522 is available for communication via a local or global network (LAN, WAN, for example via the Internet). The network adapter can be accessed by other components of the computer system 500 via the bus system 506. It is understood in this case, although it is not illustrated, that other devices can also be connected to the computer system 500.

In addition, at least parts of the training data generator system 400 for training a machine learning system with an extended set of patient data (cf. FIG. 4) can be connected to the bus system 506. The training data generator system 400 and the computer system 500 may optionally use the memories and/or the processors 402 jointly. The description of the various exemplary embodiments of the present

disclosure has been given for the purpose of improved understanding, but does not serve to directly restrict the inventive concept to these exemplary embodiments. A person skilled in the art will themselves develop further modifications and variations. The terminology used here has been selected so as to best describe the basic principles of the exemplary embodiments and to make them easily accessible to a person skilled in the art.

The principle presented here may be embodied as a system, as a method, combinations thereof and/or else as a computer program product. The computer program product may in this case comprise one (or more) computer-readable storage medium/media comprising computer-readable program instructions in order to cause a processor or a control system to implement various aspects of the present disclosure.

As media, electronic, magnetic, optical, electromagnetic or infrared media or semiconductor systems are used as forwarding medium; for example SSDs (solid state devices/drives as solid state memory), RAM (random access memory) and/or ROM (read-only memory), EEPROM (electrically erasable ROM) or any combination thereof. Suitable forwarding media also include propagating electromagnetic waves, electromagnetic waves in waveguides or other transmission media (for example light pulses in optical cables) or electrical signals transmitted in wires.

The computer-readable storage medium can be an embodying device that retains or stores instructions for use by an instruction executing device. The computer-readable program instructions that are described here may also be downloaded onto a corresponding computer system, for example as a (smartphone) app from a service provider via a cable-based connection or a mobile radio network.

The computer-readable program instructions for executing operations of the disclosure described here may be machine-dependent or machine-independent instructions, microcode, firmware, status-defining data or any source code or object code that is written for example in C++, Java or the like or in conventional procedural programming languages such as for example the programming language “C” or similar programming languages. The computer-readable program instructions may be executed in full by a computer system. In some exemplary embodiments, there may also be electronic circuits, such as, for example, programmable logic circuits, field-programmable gate arrays (FPGAs) or programmable logic arrays (PLAs), which execute the computer-readable program instructions by using status information of the computer-readable program instructions in order to configure or to individualize the electronic circuits according to aspects of the present disclosure.

The disclosure presented here is furthermore illustrated with reference to flowcharts and/or block diagrams of methods, devices (systems) and computer program products according to exemplary embodiments of the disclosure. It should be pointed out that practically any block of the flowcharts and/or block diagrams can be embodied as computer-readable program instructions.

The computer-readable program instructions can be made available to a general purpose computer, a special computer or a data processing system programmable in some other way, in order to produce a machine, such that the instructions that are executed by the processor or the computer or other programmable data processing devices generate means for implementing the functions or processes illustrated in the flowchart and/or block diagrams. These computer-readable program instructions can correspondingly also be stored on a computer-readable storage medium.

In this sense any block in the illustrated flowchart or block diagrams can represent a module, a segment or portions of instructions representing a plurality of executable instructions for implementing the specific logic function. In some exemplary embodiments, the functions represented in the individual blocks can be implemented in a different order—optionally also in parallel.

The illustrated structures, materials, sequences and equivalents of all means and/or steps with associated functions in the claims hereinafter are intended to apply all structures, materials or sequences as expressed by the claims.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method for training a machine learning system with an extended set of patient data E′/A′, the method comprising
measuring patient data as a plurality of data pairs E-A,
wherein the data pairs comprise input vectors E and assigned first output vectors A,
wherein the output vectors A represent ground truth data for a machine learning system if associated input vectors E are used during a learning phase of the machine learning system;
determining the number of data pairs of the measured patient data;
determining whether the number of data pairs lies below a previously defined training data threshold value, and if that is the case, carrying out the following steps:
selecting a physical-optical model for a relation function R on the basis of at least one first subset of the plurality of data pairs E/A,
wherein an approximation vector with respect to the associated output vector is determined by the relation function R as the result if an associated input vector E is used as the argument of the relation function R;
using at least one second subset of the plurality of data pairs E/A in order to determine corresponding second output vectors A″ from input vectors E by means of the relation function R;
determining, for all data pairs of the second subset, a respective difference vector A between the determined corresponding second output vector A″ of the relation function R and the associated ground truth data vector A;
modifying the input vectors E of the data pairs E/A of the second subset by a respective ε-vector, wherein the components of the ε-vector each have absolute values much smaller than the standard deviations of the components of the associated input vector relative to all the input vectors;
determining third output vectors A′″ of the relation function R, wherein the modified input vectors E′ of the data pairs of the second subset are used as arguments of the relation function R;
determining modified output vectors A′ by subtracting the determined difference vectors Δ from the third output vectors A′″; and
training a machine learning system for generating a machine learning model by means of the modified input vectors E′, the modified output vectors A′ and the measured patient data.

2. The method of claim 1, wherein components of the respective ε-vectors are generated randomly.

3. The method of claim 1, wherein the machine learning system is selected from the group consisting of a fully connected neural network system, a recurrent neural network system, a convolutional neural network system, a graph neural network system, a transformer network system and a foundation model system.

4. The method of claim 1, wherein a subset of the data pairs consists of biometric measurement variables of the eye and a refractive power of an intraocular lens as input vector and a refractive result of cataract surgery as output vector.

5. The method of claim 1, wherein a subset of the data pairs consists of biometric measurement variables of the eye and the refractive result of cataract surgery as input vector and the refractive power of an intraocular lens as output vector.

6. The method of claim 4, wherein the refractive result represents an objective refraction, and a subjective refraction is an additional output variable.

7. The method of claim 1, wherein a subset of the data pairs corresponds to biometric measurement variables of the eye and a refractive result of cataract surgery as input vector and an actually expected refraction as output vector.

8. The method of claim 1, wherein the relation function R is selected on the basis of a subset of the first data pairs.

9. The method of claim 1, wherein the relation function describes a physical dependence, a statistical correlation or a dependence corresponding to an individually trained machine learning system.

10. The method of claim 1, wherein the relation represents a physical-optical description of the eye.

11. The method of claim 1, wherein the components of the respective &-vector each have absolute values much smaller than or equal to standard deviations of the corresponding components of the associated input vector.

12. The method of claim 1, additionally comprising:
displaying the measured patient data;
displaying the modified input vectors E′ and the modified output vectors A′,
receiving a signal for confirming individual vector pairs of the modified input vectors E′ and the associated modified output vectors A′; and
excluding the non-confirmed vector pairs for the training of the machine learning system.

13. A training data generator system for training a machine learning system with an extended set of patient data E′/A′, the system comprising:
a processor; and
a memory which operatively cooperates with the processor to store instructions which, when executed by the processor, cause the processor to:
measure patient data as a plurality of data pairs E/A,
wherein the data pairs comprise input vectors E and assigned first output vectors A, and
wherein the output vectors A represent ground truth data for a machine learning system if associated input vectors E are used during a learning phase of the machine learning system;
determine the number of data pairs of the measured patient data;
determining whether the number of data pairs lies below a previously defined training data threshold value, and if this is the case, carrying out the following steps:
select a physical-optical model for a relation function R on the basis of at least one first subset of the plurality of data pairs E/A,
wherein an approximation vector with respect to the associated output vector is determined by the relation function R as the result if an associated input vector E is used as the argument of the relation function R;
use at least one second subset of the plurality of data pairs E/A in order to determine corresponding second output vectors A″ from input vectors E by means of the relation function R;
determine, for all data pairs of the second subset, a respective difference vector Δ between the determined corresponding second output vector A″ of the relation function R and the associated ground truth data vector A;
modify the input vectors E of the data pairs E/A of the second subset by a respective ε-vector, wherein components of the ε-vector each have absolute values much smaller than standard deviations of the corresponding components of the associated input vector;
determine third output vectors A′″ of the relation function R, wherein the modified input vectors E′ of the data pairs of the second subset are used as arguments of the relation function R;
determine modified output vectors A′ by subtracting the determined difference vectors A from the third output vectors A′″; and
train a machine learning system for generating a machine learning model by means of the modified input vectors E′, the modified output vectors A′ and the measured patient data.

14. The system of claim 13, wherein components of the respective ε-vectors are generated randomly.

15. The system of claim 13, wherein the machine learning system is selected from the group consisting of a fully connected neural network system, a recurrent neural network system, a convolutional neural network system, a graph neural network system, a transformer network system and a foundation model system.

16. The system of claim 13, wherein a subset of the data pairs consists of biometric measurement variables of the eye and a refractive power of an intraocular lens as input vector and a refractive result of cataract surgery as output vector.

17. The system of claim 13, wherein a subset of the data pairs consists of biometric measurement variables of the eye and the refractive result of cataract surgery as input vector and the refractive power of an intraocular lens as output vector.

18. The system of claim 17, wherein the refractive result represents an objective refraction, and a subjective refraction is an additional output variable.

19. The system of claim 13, wherein a subset of the data pairs corresponds to biometric measurement variables of the eye and a refractive result of cataract surgery as input vector and an actually expected refraction as output vector.

20. The system of claim 13, wherein the relation function R is selected on the basis of a subset of the first data pairs.

21. The system of claim 13, wherein the relation function describes a physical dependence, a statistical correlation or a dependence corresponding to an individually trained machine learning system.

22. The system of claim 13, wherein the relation represents a physical-optical description of the eye.

23. The system of claim 13, wherein the components of the respective ε-vector each have absolute values much smaller than or equal to standard deviations of the corresponding components of the associated input vector.

24. The system of claim 13, wherein the processor is also enabled to:
facilitate displaying the measured patient data;
facilitate displaying the modified input vectors E′ and the modified output vectors A′;
receive a signal for confirming individual vector pairs of the modified input vectors E′ and the associated modified output vectors A′; and
exclude the non-confirmed vector pairs for the training of the machine learning system.

25. A computer program product for training a machine learning system with an extended set of patient data, wherein the computer program product comprises a computer-readable storage medium comprising program instructions stored thereon, wherein the program instructions are executable by one or more computers or control units and cause said one or more computers or control units to:
measure patient data as a plurality of data pairs E-A,
wherein the data pairs comprise input vectors E and assigned first output vectors A, and
wherein the output vectors A represent ground truth data for a machine learning system if associated input vectors E are used during a learning phase of the machine learning system;
determine the number of data pairs of the measured patient data;
determine whether the number of data pairs lies below a previously defined training data threshold value, and if that is the case, carrying out the following steps:
select a physical-optical model for a relation function R on the basis of at least one first subset of the plurality of data pairs E/A,
wherein an approximation vector with respect to the associated output vector is determined by the relation function R as the result if an associated input vector E is used as the argument of the relation function R;
use at least one second subset of the plurality of data pairs E/A in order to determine corresponding second output vectors A″ from input vectors E by means of the relation function R;
determine, for all data pairs of the second subset, a respective difference vector A between the determined corresponding second output vector A″ of the relation function R and the associated ground truth data vector A;
modify the input vectors E of the data pairs E/A of the second subset by a respective ε-vector, wherein the components of the ε-vector each have absolute values much smaller than the standard deviations of the components of the associated input vector relative to all the input vectors;
determine third output vectors A′″ of the relation function R, wherein the modified input vectors E′ of the data pairs of the second subset are used as arguments of the relation function R;
determine modified output vectors A′ by subtracting the determined difference vectors Δ from the third output vectors A′″; and
train a machine learning system for generating a machine learning model by means of the modified input vectors E′, the modified output vectors A′ and the measured patient data.
